SEQUENCE LISTING (15 sequences)

SEQ ID NO: 1 - DNA, 675 bp, Lonomia obliqua
ggatccgacgtagttatagatggtgcgtgtcctgacatgaaggcggtatcgaaatttgacatgaatgcttatcaaggaacgtggtacgagatcaagaaattccccgtggctaatgaagcgaacggtgattgtggaagtgttgagtatacccccgacaatggactactgaaggtgagagcgggacacgttgaagatgatatcgagaagtttgttgtcggagtcctcaccaagaatgcagacaccagcgatgctgagctcactctcagcgttgtagtcggcgactacgtccgcgttgcaccgctgtggattctttctactgattacgacaactatgctatcggctactcctgcaaagactacaagaagagcaaccaacacagggtaaacatctggattctctcgaggaccaagactctcaacgaaagttccaagtccactgtcaacaagttccttaaggagcactcaaaggagttcgatcaatcgaaatttgtcgagacagatttctccgaaaaagcatgcttcttcaagaaatcacacgtgtacactgtaccattcggagcttaaattcgatttgtttggtctagtgctaataaaaggtttttgggtttttaaatttaattaaccttataagtggaattaataataaataagtgaaacaaaaaaaaaaaaaaaaaa

SEQ ID NO: 2 - protein, 187 aa, Lonomia obliqua
GSDVVIDGACPDMKAVSKFDMNAYQGTWYEIKKFPVANEANGDCGSVEYTPDNGLLKVRAGHVEDDIEKFVVGVLTKNADTSDAELTLSVVVGDYVRVAPLWILSTDYDNYAIGYSCKDYKKSNQHRVNIWILSRTKTLNESSKSTVNKFLKEHSKEFDQSKFVETDFSEKACFFKKSHVYTVPFGA

SEQ ID NO: 3 - DNA, 28 bp, Artificial Sequence
  Primer
atggatccgacgtagttatmgayggngc

SEQ ID NO: 4 - protein, 8 aa, Artificial Sequence
  Synthetically generated peptide
IDVVIDGA

SEQ ID NO: 5 - DNA, 28 bp, Artificial Sequence
  Primer
atggatccgcttaccarggaacttggta

SEQ ID NO: 6 - protein, 8 aa, Artificial Sequence
  Synthetically generated peptide
IAYQGTWY

SEQ ID NO: 7 - protein, 150 aa, Lonomia obliqua
AYQGTWYEIKKFPVANEANGDCGSVEYTPDNGLLKVRAGHVEDDIEKFVVGVLTKNADTSDAELTLSVVVGDYVRVAPLWILSTDYDNYAIGYSCKDYKKSNQHRVNIWILSRTKTLNESSKSTVNKFLKEHSKEFDQSKFVETDFSEKA

SEQ ID NO: 8 - protein, 149 aa, Pieris brassicae
NYHGKWWEVAKYPNSVEKYGKCGWAEYTPEGKSVKVSNYHVIHGKEYFIEGTAYPVGDSKIGKIYHKLTYGGVTKENVFNVLSTDNKNYIIGYYCKYDEDKKGHQDFVWVLSRSKVLTGEAKTAVENYLIGSPVVDSQKLVYSDFSEAA

SEQ ID NO: 9 - protein, 143 aa, Rattus norvegicus
KLNGDWFSIVVASNKREKIEENGSMRVFMQHIDVLENSLGFKFRIKENGECRELYVAYKTPEDGEYFVEYDGGNTFTILKTDYDRYVMFHLINFKNGETFQLMVLYGRTKDLSSDIKEKFAKLCEAHGITRDNIIDLTKTDRC

SEQ ID NO: 10 - protein, 143 aa, Rattus norvegicus
DVSGTWYLKAAAWDKEIPKKFGSVSVTPMKIKTLEGGNLQVKFTVLIAGRCKEMSTVLEKTDEPAKYTAYSGKQVLYIIPSSVEDHYIFYYEGKIHRHHFQIAKLVGRDPEINQEALEDFQSVVRAGGLNPDNIFIPKQSETC

SEQ ID NO: 11 - protein, 144 aa, Homo sapiens
KYLGRWYEIEKIPTTFENGRCIQANYSLMENGKIKVLNQELRADGTVNQIEGEATPVNLTEPAKLEVKFSWFMPSAPYWILATDYENYAVYSCTCIIQLFHVDFAWILARNPNLPPETVDSLKNILTSNNIDVKKMTVTDQVNC

SEQ ID NO: 12 - protein, 151 aa, Manduca sexta
AFAGAWHEIAKLPLENENQGKCTIAEYKYDGKKASVYNSFVSNGVKEYMEGDLEIAPDAKYTKQGKYVMTFKFGQRVVNLVPWVLATDYKNYAINYNCDYHPDKKAHSIHAWILSKSKVLEGNTKEVVDNVLKTFSHLISKFISNDFSEAA

SEQ ID NO: 13 - protein, 145 aa, Homarus gammarus
RYAGRWYQTHIIENAYQPVTRCIHSNYEYSTNDYGFKVTTAGFNPNDEYLKIDFKVYPTKEFPAAHMLIDAPSVAAPYEVIETDYETYSCVYSCITTDNYKSEFAFVFSRTPQTSGPAVEKTAAVFNKNGVEFSKFVPVSHTAEC

SEQ ID NO: 14 - protein, 139 aa, Homarus gammarus
SYAGVWYQFALTNNPYQLIEKCVRNEYSFDGKQFVIKSTGIAYDGNLLKRNGKLYPNPFGEPHLSIDYENSFAAPLVILETDYSNYACLYSCIDYNFGYHSDFSFIFSRSANLADQYVKKCEAAFKNINVDTTRFVKTV

SEQ ID NO: 15 - protein, 157 aa, Gallus gallus
RYAGKWYALAKKDPEGLFLQDNISAEYTVEEDGTMTASSKGRVKLFGFWVICADMAAQYTVPDPTTPAKMYMTYQGLASYLSSGGDNYWVIDTDYDNYAITYACRSKEDGSCDDGYSLIFSRNPRGLPPAIQRIVRQKQEEICMSGQFQPVLQSGAC

(19) United States
(12) Patent Application Publication
(10) Pub. No.: US 2008/0267944 A1
(43) Pub. Date: Oct. 30, 2008

(54) Process for Obtaining Recombinant Prothrombin Activating Protease (Rlopap) in Monomeric form; the Recombinant Prothrombin Activating Protease (Rlopap) as Well as its Amino Acid Sequence; the Use of this Protease as a Defibrinogenase

(75) Inventors: Ana Marisa Chudzinski-Tavassi, Sao Paulo (BR); Cleyson Valenca Reis, Sao Paulo (BR); Paulo Lee Ho, Sao Paulo (BR); Celso Romero Ramos, Sao Paulo (BR)

Correspondence Address: FISH & RICHARDSON PC, P.O. BOX 1022, MINNEAPOLIS, MN, 55440-1022, US

(73) Assignees: Biolab Sanus Famaceutica Ltda., Sao Paulo (BR); Fundacao De Amparo A Pesquisa Do Estado De Sao Paulo - Fapesp, Sao Paulo (BR)

(21) Appl. No.: 11/574,213

(22) PCT Filed: Aug. 24, 2005
(86) PCT No.: PCT/BR05/00171

§ 371 Date: Apr. 11, 2008

(30) Foreign Application Priority Data
Aug. 24, 2004 (BR) PI0403882-7

(51) Int. Cl. A61K 38/43; C12N 15/11; C12N 9/50; A61P 7/00; C12P 21/04; C12Q 1/37

(52) U.S. Cl. 424/94.63; 536/23.2; 435/219; 435/69.1; 435/23

(57) ABSTRACT

This invention refers to the process for obtaining the recombinant prothrombin activating protease (rLopap) in monomeric form, the recombinant prothrombin activating protease (rLopap), as well as its amino acid sequence. In addition to that, this invention also refers to the use of this protease for depleting the blood fibrinogen, and serve as diagnosis kit for dysprothrombinemias. This invention describes the obtainence in recombinant form and the characterization of a prothrombin activator protease of 21 kDa, named rLopap (Lonomia obliqua prothrombin activator protease), with serineproteases characteristics however it shows sequence of conserved amino acids as in a lipocalin family. The protein presents pro-coagulating activity, depleting blood fibrinogen and prolonging the coagulation time of human blood/plasma. The obtainence of rLopap in its recombinant form and showing adequate activity for allowing clinical Pharmacology essays is presented in this invention.

### STATEMENT OF THE OBJECT OF THE INVENTION

This invention refers to the process for obtaining the recombinant prothrombin activating protease (rLopap) in monomeric form; the recombinant prothrombin activating protease (rLopap) as well as its amino acid sequence; the use of this protease for depleting blood fibrinogen and the diagnosis kit for dysprothrombinemias.

### BACKGROUND OF THE INVENTION

The Lonomia genus is known for causing a systemic envenoming as a consequence of its venom inoculation through the skin, presenting hemorrhagic manifestations of variable intensity, sometimes casing the death of the exposed subject (Lorini, 1999). The Walker Lonomia obliqua species (Lemaire, 1972) has caused epidemic dimension accidents since 1989 in restricted areas in the south of Brazil (Rio Grande do Sul, Santa Catarina and Paraná) (BRAZIL, 1998).

The exposed patients, among other symptoms, show mainly, blood dyscrasia signs (alteration in the proportion of the blood elements) after a period that may vary from 1 to 48 hours, followed or not by hemorrhagic manifestations and could even resulting in death (Kelen et al, 1995; Brazil, 1998).

Recently, Zannin et. al. determined the coagulation parameters and the plasma fibrinolysis of 105 patients and verified, corroborating with some existing data, that the accident affects the mechanisms of coagulation and fibrinolysis. These results indicate an intense consumption coagulopathy that can be attributed to components of the venom present in the bristles of the Lonomia obliqua caterpillar, with powerful procoagulant action and secondary activation of the fibrinolysis (Zannin et. al., 2002).

The Lonomia obliqua caterpillar venom presents some components that interfere in the coagulation system. In the L. obliqua bristles extract the presence of prothrombin activators and of Factor X was already described (Donato et. al., 1998; Kelen et. al., 1995).

The inventors of this patent requesting isolated and characterized a prothrombin activating protease of 69 kDa, named Lopap (Lonomia obliqua prothrombin activator protease). It has serineprotease characteristics and procoagulant activity in rats, depleting blood of fibrinogen and altering in only 30% the number of platelets, although completely inhibiting the aggregation function of the platelets induced by collagen for increasing the PGI2 levels.

Lopap, when injected in rats per intraperitoneal administration, develops thrombi in venules and arterioles, causing polymorphonuclear migration to the lungs and kidneys (Reis et. al., 1999, Reis et al, 2001 a, b).

Recently it could be verified that Lopap acts in endothelial cells (HUVECs), as an inductor of the adhesion molecules expression like ICAM-1 and E-selectin, however it does not express VCAM. It does also induce the increase of IL-8 and of PGI₂. The non-expression of VCAM suggests that the pro-inflammatory action of Lopap can not be compared to the TNF-α or to the thrombin on endothelial cells. High concentrations of PGI2 can also be acting as platelet anti-aggregating.

It was also verified that the thrombin produced by Lopap is functional and inhibited by Antithrombin III (AT) and it is able to aggregate platelets as well as to coagulate plasma and fibrinogen, suggesting to be similar to the α-thrombin (Chudzinski-Tavassi et al, 2001).

The L. obliqua bristles extract is effective in the experimental prevention of venous thrombosis in rats (Prezoto et. al, 2002), justifying the studies using purified venom fractions for elucidating the mechanisms of this effect.

During this study, a cDNA library in pGEM11zf+plasmids was built and through the use of specific degenerated initiator oligonucleotides obtained from the N-terminus portion of the native protein, a clone, correspondent to Lopap was isolated and its sequence was determined. The sequence of amino acids deduced from the cDNA was aligned with other sequences of the CLUSTAL W program from the “GenBank”.

Lopap presented 37% of identity with BBP BILIN-BINDING PROTEIN of Pieris brassicae; 34% with ApoD —APOLIPOPROTEIN D PRECURSOR of Homo sapiens; 35% with INS-A —INSECTICYANIN A of Manduca sexta; 22% with CC-A2—A2 CRUSTACYANIN A2 SUB-UNITY of Homarus gammarus; 26% with CC-C1.—C1-CRUSTACYANIN C1 SUB-UNITY of Homarus gammarus, and 27% with PURP.—PURPURIN PRECURSOR of Gallus gallus (gi 131549).

The lipocalins (from the Greek “lipos”=fat and “calyx”=goblet) are part of a large group, presented in several species, always expanding and showing a great functional and structural variety. In general they are small proteins varying between 160 and 180 amino acids, presenting common characteristics (Flower et. al, 2000).

These proteins show few similarities one another (around 20%), however they contain two highly conserved domains (eight amino acids residues bound by hydrogen bridges to a “β-barrel” region) allowing to classify them as lipocalins. These regions are responsible for the high similarity of its secondary and tertiary structures.

The lipocalins are able to bind themselves to the molecules (especially the hydrophobic as retinol) for presenting a hydrophobic “β-barrel”-kind region, composed by a cavity inside and a loop outside and containing a binding site to different ligands. The diversity of these cavities and “loops” bring to each protein the possibility of accommodating ligands of different sizes, forms and chemical characteristics.

From the sequence obtained by cDNA, an approach for studying the Lopap structural model using the Swiss PDB Viewer 3.7 (b2) program and the Swiss Model Server was conducted. What was found is a typical lipocalin family structure, however it was not possible to visualize a serine protease site through this modeling.

Lipocalin family member proteins were not described in literature as having function of a prothrombin activating protease. Different from other activators of animal venoms, rLopap hydrolyses the prothrombin, generating fragments (prethrombin 2, thrombin and fragments 1.2 and fragments 1 and 2), independently of its prothrombinase complex components. This kind of fragmentation promotes a slower formation of thrombin, enabling a better control of it and impeding its action. Moreover, for promoting cellular responses concerning the endothelial level as the expression of NO, PGI2, rLopap is different from other known activators since it can modulate platelet aggregation response. Besides that, this protein has the advantage to be easily obtained in its recombinant form, when compared to other venom prothrombin activators.

Based on the rLopap capacity to activate prothrombin, a dosage kit of these factor concentrations in the plasma can be prepared. After the rLopap incubation with the diluted human plasma, the thrombin formed is determined by the hydrolyzes of a specific chromogenic or fluorogenic substrate (on the market, for instance S2238 of Chromogenix presenting the sequence H-D-phenylalanyl-L-pipecolyl-L-arginyl-p-nitroanilide or that can be synthesized as the case of Abz-FFNPRTFGSGQ-EDDnp). When referring to the absorbency of chromogenic substrates, measured in 405 nm, it shows to be proportional to that of the sample prothrombin activity and it is comparable to the standard human plasma curve on the market.

Based on the defibrinogenase potential of the protein activating activity without altering the platelet number and in the structural differences that allow considering Lopap as a new prothrombin activator, a patent request was deposited by the same authors, concerning this protein —PI 02002698.

The PI 0200269-8, in general lines, describes a purifying process of soluble proteins from the bristles of the Lonomia obliqua caterpillar with prothrombin activating activity, a process for the partial determination of the amino acids sequence of the referred prothrombin activator, a process for determining this prothrombin activating activity of fraction II as well as the prothrombin activator and the use of this activator.

Following the same research approach toward the protein mentioned above, the inventors are now presenting the Lopap in its recombinant form and with adequate activity for allowing clinical Pharmacology essays.

According to this invention, Lopap was subcloned in expression vector and expressed in E. coli.

For conducting this invention the following items were used:

Lineage: Escherichia coli;
1) Strain DH5α: ø80 dlacZΔM15, recA1, endA1, gyrA96, thi-1, hsdR17 (rₖ⁻, mₖ⁺) SupE44, relA1, deoR Δ(lac ZYA-arg I) u169.
2) Strain BL21 (DE3): F′, ampT, hsdS_{B}(r₈⁻, m₈⁻), dcm, gal (DE3). The DE3 bacteriophage contains the RNA polymerize gene of the T7 phage under the control of the Lac UV5 promoter, inducible by isopropil-thio-β-galactoside (IPTG).

Plasmids:

1) pGEM-11Zf (+): vector digested with EcoRI e Not I used in the plasmid library construction. PROMEGA Technical Manual, 1999.
2) “easy” pGEM-T: The “easy” pGEM-T plasmid contains the T7 and SP6 promoters flanked the MCS (“multiple cloning site”) region for sub-cloning the PCR products. PROMEGA Technical Manual, 1999.
3) pAE: expression vector derived from pRSETA (Invitrogen) and from pET3-His (Chen, 1994) constructed in the Molecular Biotechnology Laboratory of the Institute Butantan (Ramos et. al, 2003).

The pAE is a high expression vector that combines the T7 promoter efficiency and the high number of pRSETA plasmid copies with an N-terminus fusion of six histidines non-removable from pET3-His, allowing the purification of recombinant proteins through the IMAC (“Immobilized Metal Affinity Chromatography”). The addition of this small fusion does not interfere in the activity of the majority of the studied recombinant proteins.

For the Synthesis of the initiator oligonucleotides in PCR reactions, the process was conducted as follows:

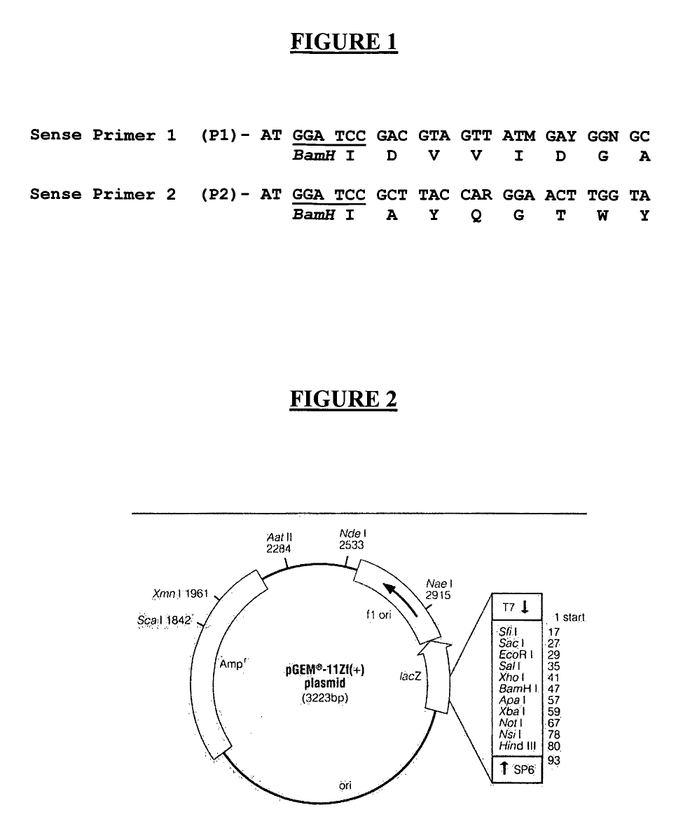

The “sense” oligonucleotides (P1 and P2) were obtained from the N-terminus sequence of the protein (FIG. 1). A BamHI restriction site for subsequent unidirectional cloning was preferentially added to these oligonucleotides. The “sense” and “anti-sense” oligonucleotides were also used. All of them were diluted in TE (Tris-HCl 10 mM, EDTA 1 mM) buffer for a final concentration of 10-pmol/μl (100 μM).

For the mRNA preparation, the procedures were defined as follows:

### Extracting and Preparing the Bristles

The caterpillars were anesthetized in CO₂ (dry ice) condition and their spicules were cut (2.7 g) and placed in a sterile plastic tube previously weighed, immerse in liquid nitrogen. The spicules were grounded in a mortar, after being treated with DEPC (diethyl pyrocarbonate) for eliminating RNAses, using dry ice and liquid nitrogen until turning into a fine powder.

### Extracting the Total RNA

The spicule powder was used for obtaining the total RNA using preferably the Triazol method in accordance with the methodology described in the manual of its manufacturer.

### Eletrophoretic Profile of the Total RNA

The accessories of the electrophoresis system were treated with hydrogen peroxide (H₂O₂) 3%, for eliminating RNAses and were washed with sterilized DEPC treated water. A 1.5% agarose gel in 10 mM, pH 7.0 sodium phosphate buffer was deposited in the regular system. Two samples containing 10 or 15 μl of total RNA (16.7 ng/μl), 5 μl of sample buffer and DEPC treated H₂O for a final volume of 25 μl, were applied in the gel. The sample migration was conducted at 5 V/cm until bromophenol would reach ⅔ of the gel.

Purification of the mRNA in Oligo (dT) Cellulose Affinity Column

The mRNA was purified in oligo dT cellulose affinity column washed with NaOH 0.1 N and balanced with 1 ml of Tris-HCl 10 mM, EDTA 1 mM, NaCl 300 mM, SDS 0.1%, pH 7.0 buffer.

3 ml of this buffer was added to the total RNA followed by the incubation at 70° C. for 5 minutes, cooling it in ice for another 5 minutes and applied in the affinity column. The column was drained by gravity and washed using more 4 ml of the buffer for eliminating every RNA that was not a mRNA. The mRNA was eluded with 1.5 ml of Tris-HCl 10 mM, EDTA 1 mM, SDS 0.1%, pH 7.0 buffer and collected in clean treated tube, heated at 70° C. for 5 minutes and cooled in ice for another 5 minutes. After incubating the material for 20 minutes at room temperature, 90 μl of NaCl 5 M was added to it and again it was applied in the column re-balanced with the buffer. What was obtained after another washing using 4 ml of the buffer and eluting the material with 1.5 ml of elution buffer, was precipitated “overnight” with 90 μl of NaCl 5 M and 3 ml of absolute ethyl alcohol at −80° C. The material was then centrifuged in 7000 g for 20 minutes at 4° C. and the supernatant was rejected. The mRNA was washed with 1 ml of ethyl alcohol 75% and centrifuged in 7000 g for 2 minutes at 4° C.

After drying it, the precipitate mRNA was re-suspended in 20 μl of DEPC treated H₂O and maintained at −80° C.

The mRNA Quantification

For a final volume of 500 μl, 2 μl of mRNA in 498 μl of sterilized H₂O milli-Q was added to it. The optical density readings were conducted in 260 and 280 nm in quartz cuvets of 500 μl. The mRNA concentration was calculated using the equation:

[RNA]=A₂₆₀×D×40μg/ml
Where D=the dilution factor

Concerning the construction of the cDNA library, the following procedures were performed:

The cDNA library was constructed from 4.0 μg of isolated mRNA, using preferably the SuperScrip™ Plasmid System for cDNA Synthesis and Plasmid Cloning (Life Technologies) modified kit.

### Synthesis of the First STRAIN

4.0 μg of mRNA was diluted in 6 μl of DEPC treated H₂O in which 1.5 μl of NotI adaptation primer was added and then it was heated at 70° C. for 10 minutes, cooled in ice bath and quickly centrifuged. 4 μl of first strain buffer 5×, 2 μl of DTT 0.1 M, 1 μl of mixture of dNTP 10 mM and 0.5 μl of H₂O were added to the tube. The reaction was homogenized, quickly centrifuged and balanced at 44° C. for 2 minutes. 5 μl of Super Script II RT enzyme was added and the mixture was incubated at 44° C. for more 90 minutes. A 4° C.-cooling process interrupted the reaction.

### Synthesis of the Second Strain

It was added to the mixture of the first strain reaction, 91 μl of H₂O, 30 μl of the second strain buffer, 3 μl of dNTP 10 mM mixture, 1 μl of E. coli DNA ligase 10 U/μl, 4 μl of E. coli DNA polymerase I 10 U/ml and 1 μl of E coli RNAase H (2 U/μl). After gently swirling the mixture, it was incubated at 16° C. for 2 hours and added to it 2 μl of T4 DNA polymerase I conducting more 5 minutes of incubation at the same temperature. The reaction was interrupted by cooling process in ice and by adding 10 μl of EDTA 0.5 M.

### Screening of Fragments Sizes in Agarose Gel

All the reaction in the second strain added with 17 μl of Ficoll xylene cyanol free was applied in agarose gel 1% and after 1 cm of sample migration in 80 V in electrophoresis system two bands were cut out of the gel. One containing fragments between 400 and 800 pb (low molecular weight—BA) and the other with fragments over 800 pb (high molecular weight—BB).

The DNA was purified from the gel using preferably the Concert Gel Extraction Systems (Life Technologies) kit, the cDNA eluded with 50 μl of H₂O heated at 65° C. and reduced into 30 μl using a concentration process.

Binding cDNA to the EcoRI Adaptors

It was added to the reaction tube, 10 μl of T4 DNA ligase 5× buffer, 5 μl of Eco RI (Amersham) adaptors and 5 μl of T4 DNA ligase with posterior incubation at 16° C. for 16 hours. Right after that, the reaction was heated at 65° C. for 10 minutes and cooled in ice. After adding 2 μl of the ATP solution and 2 μl of the T4 polymerase kinase, the reaction was incubated for 30 minutes at 37° C.

The cDNA was extracted using 55 μl of phenol/chloroformium/alcohol isoamilic (25:24:1), swirled and centrifuged in 14000 g for 5 minutes at room temperature. The superior aqueous phase was transferred to another tube and added with 2 volumes of absolute ethanol and 1 volume of 3-M sodium acetate and cooled at −80° C. for 1 hour. After centrifuged in 14000 g for 20 minutes and washed with 500 μl of ethanol 70%, the cDNA was dried in flow for about 5 minutes.

Digestion with NotI

It was added to the precipitated, 41 μl of H₂O, 5 μl of REact reaction buffer, 4 μl of NotI, gently homogenized and followed by incubation for 2 hours at 37° C.

### Second Size Screening in Agarose Gel

50 μl of the reactions (of high and low molecular weight fragments) were applied in agarose gel 1% and after electrophoresis, the bands were cut out of the gel. The cDNAs of high and low weights were purified and eluded with 50 μl of H₂O as described in the first size screening (Screening of fragments sizes in agarose gel).

Unidirectional Binding from the cDNA to the pGEM11Zf(+) Vector

The two fractions (14 μl), of high and low weights were added with 4 μl of T4 DNA ligase buffer, 1 μl of clonage vector, preferentially pGEM11Zf(+) (previously digested with the EcoR I—Not I enzymes) (FIG. 2) and 1 μl of T4 DNA ligase and incubated at 16° C. for 18 hours.

The bacterial transformation process of this invention follows the procedure described below:

### Transformation of Competent E. Coli DH5α

DNAs of high and low weights (2 μl) were added to 50 μl of calcium competent bacteria (DH5α) prepared in accordance with the Inoue et al. (1990) method being maintained at −80° C. and previously defrosted in ice for 15 minutes. The solutions were incubated for 30 minutes in ice and afterwards submitted to a heating shock of 42° C. for 2 minutes and again in ice for 5 minutes.

350 μl of SOC medium was added to the transformed bacteria and then transferred to aerated tubes being incubated at 37° C. under swirling conditions (220 rpm/min) for 90 minutes. 200 μl of cDNA of high and low molecular weights was placed in plates with 2YT-ampicillin medium. These plates were incubated for 18 hours at 37° C. 20 colonies containing high weight inserts and 20 containing low weight ones, were incubated in two plates at 37° C. in 2.5 ml of 2YT-ampicilina 100 μg/ml medium for 18 hours under swirling condition of 200 rpm. The cDNAs were purified using preferably the mini-prep Concert Rapid Plasmid (Life Technologies) kit eluded with 50 μl of TE at 65° C.

For analyzing the library concerning the plasmids, the following procedure was taken:

The plasmids (4 μl) were digested at 37° C. for 2 hours in presence of 1 μl of specific reaction buffer, 4 μl of water, 0.5 μl of EcoRI (10 U/μl) enzyme. The 0.5 μl of HindIII (10 U/μl) and the fragments generated were analyzed in agarose gel 1% with ethidium bromide. All the analyzed plasmids were submitted to sequencing process.

Aiming to obtain the amplification of the library, mixtures containing 50 μl of DH5α calcium-competent bacteria and 5 μl of high or low molecular weight (BA or BB) DNA bound to the clonage vector were incubated for 30 min in ice, for 2 min at 42° C. After that, they were returned in ice for another 5 min. After that, 10 ml of 2YT/ampicilina 100 μg/ml medium was added to them. These solutions were gently homogenized and aliquots of 2.5 ml were transferred by pipettes to deaerated tubes and incubated at 37° C. for 18 h. After that the plasmidial DNA was extracted using mini-preps columns, eluded with 50 μl of H₂O at 65° C. and maintained at −20° C.

### The Polymerase Chain Reaction (PCR)

The PCRs “Polymerase Chain Reaction” prepared for a 50-μl final volume contained 1 μl of dNTPs 10 mM, 5 μl of Buffer for Taq DNA polymerase 10×, 1.5 μl of MgSO₄ 50 mM and 0.5 μl of TaqDNA polymerase 2.5 U. For amplifying the cDNA that codifies for the prothrombin activating protein, 411 of amplified plasmidial DNA, 4 μl of oligonucleotide P1 10 pM and 2 μl of oligonucleotide SP6 10 pM were used. The reaction was incubated in a thermocycling device in which a denaturation program was conducted initially at 94° C. for 3 min, 30 denaturation cycles (94° C. for 45 seconds), annealing (50° C. for 25 seconds), extension (72° C. for 4 min) and a final extension at 72° C. for 15 min. After that, the samples were applied in agarose gel 1%. After 2 h of electrophoresis migration in 80 V, the bands corresponding to the expected amplification of the products were cut out of the gel and the DNA was extracted and eluded in 30 μl of H₂O for binding to a second clonage vector, preferably the “pGEM-T Easy Vector Systems” (PROMEGA).

For performing the binding of the DNA to the “easy” pGEM-T, the following methodology was chosen:

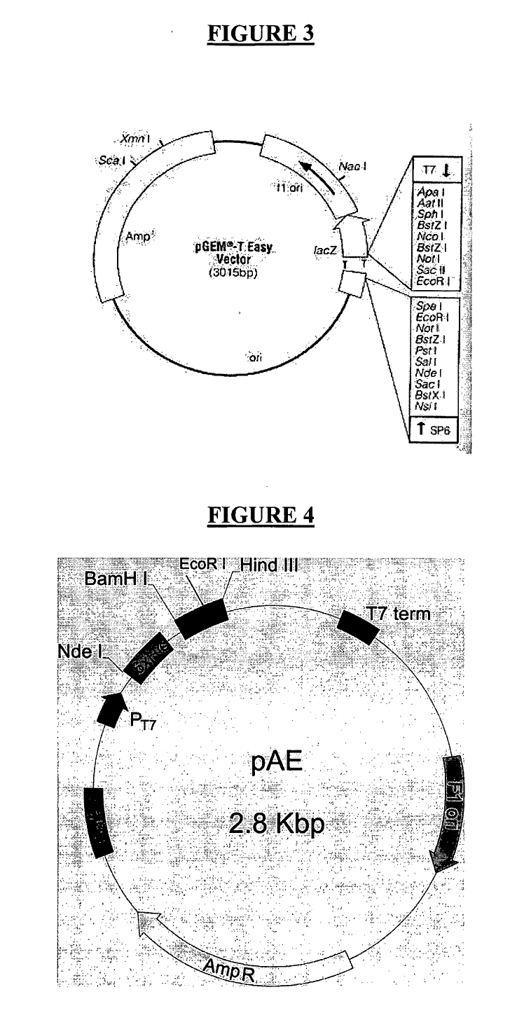

The bindings were conducted for a final volume of 10 μl containing 6 μl of the product PCR (1700 pb), 1 μl of the pGEM-T vector (FIG. 3), 2 μl of T4 DNA ligase 5× buffer and 1 μl of the T4 DNA Ligase 1 U/μl at 16° C. for 18 hours.

The relation vector:insert was calculated in accordance with the following equation:

$$\frac{X \times I}{V} \times R = Y$$

### Where:

X=ng of vector; I=Kb of the insert; R=molar relation insert/vector; Y=ng of insert; V=Kb of the vector.

Strains of DH5α E. coli were incubated with 5 μl of the vector-insert binding reaction and placed in plates. Out of the formed colonies, 40 were collected for pre-inoculum and “mini-prep” procedures exactly as described in the protocol for the transformation of DH5α-competent E. coli.

### The Selection of the Recombinant Plasmids in Agarose Gel

Before processing the “mini-preps” reactions, 300 μl of each pre inoculum were submitted to a quick process of phenol:chloroformium purification in accordance with the method described by Beuken et al. (1998). After that, 20 μl of each sample were applied in agarose gel 1%, in TAE 1× buffer. After the electrophoresis running, the gel was stained with ethidium bromide solution of 0.1 μg/ml for screening the larger recombinant plasmids, under UV light (Sambrook, 1989). The positive clones of the previous item were submitted to the “mini-preps” and eluded with 60 μl of water.

### PCR Using Primer 2 (P2)

PCR reactions were prepared for a final volume of 10 μl containing 0.2 μl of dNTPs 10 mM, 1.0 μl of buffer for Taq DNA polymerase 10×, 0.3 μl of MgSO₄ 50 mM, 0.1 μl of Taq DNA polymerase 5 U/μl. For the cDNA amplification, 2 μl of the positive clones purified in the previous item were used, in dilution of 1/50, as templates and 0.8 μl of oligonucleotide P2 10 pM and 0.4 μl of SP6 10 pM as primers. The reaction was incubated in a Perkin-Elmer thermocycling device of model 9600, conducting a initial denaturation program at 94° C. for 3 min, 30 cycles of denaturation (94° C. for 45 seconds), annealing (50° C. for 25 seconds), extension (72° C. for 4 min) and a final extension at 72° C. for 15 min. After that, the samples were applied in agarose gel 1%.

Plasmidial DNA Digestion with Restriction Enzymes

The DNAs of the purified clones amplified by PCR using primer P1 and P2, were digested at 37° C. for 2 h in a solution containing 5 μl of the plasmidial DNA, 2 μl of specific reaction buffer, 0.5 μl of Hind III (10 U/μl), 0.5 of BamH I (10 U/μl) and 12 μl of H₂O for a final volume of 20 μl.

The same plasmidial DNAs (5 μl) were incubated in same conditions with 1 μl of specific reaction buffer, 1 μl of EcoR I (10 U/μl) and 12 μl of water.

The digestion products were analyzed in agarose gel 1%. The clones of the library and the DNAs subcloned in “easy” pGEM-T were sequenced.

For performing the sequencing of the DNAs the method of chain termination by dideoxynucleotide was chosen, adapting it to the automatic sequencing process. 400 ng of the plasmidial DNA was prepared through the purification by mini-preps, which was used as molds in the sequencing reaction. T7 and SP6 were used in the described oligonucleotides reactions. After the thermocycling, the amplification products were separated in the DNA gel sequencing of 36 cm of length (4.25% acrylamide:bis-acrylamide in a proportion of 19:1, in 1×TBE and 7 M Urea. The detection system of this device consists in a laser source and a fluorescence detector, set at the lower part of the sequencing gel. Each dNTP emits a specific fluorescence recognized by the detector that sends the message for a computer that will automatically register the position of the nucleotide in the electropherogram. The running was conducted for 7 hours. All the sequenced DNAs were compared with the “GenBank” sequences through the site www.ncbi.nlm.nih.gov/, based on the algorithm of the BLASTx and BLASTn programs, or site www.ebi.ac.uk/ for the FASTA program.

The expression process of the recombinant protein, preferably E. coli strain BL21 (DES), used in this invention follows the procedure lines as listed below:

### Binding to the pAE Vector

The positive clones in which sequenced inserts confirmed the Lopap sequence, were incubated in 7 ml of LB/ampicillin at 37° C. for 18 hours, and afterwards they were submitted to the mini-preps and eluded with 50 μl of water.

The purified DNAs were digested at 37° C. for 5 h in a solution containing 20 μl of plasmidial DNA, 5 μl of specific reaction buffer, 1.0 μl of EcoR I (10 U/μl), 1.0 μl of BamH I (10 U/μl) and 23 μl of H₂O for a final volume of 50 μl.

After electrophoresis in preparative agarose gel 1%, the bands with around 600 pb were purified from the gel, eluded with 30 μl of H₂O and dried by vacuum at 45° C. for 1 h.

The plasmid was re-suspended in 10 μl of H₂O and 3.5 μl of it was incubated at 16° C. for 18 h with 3.5 μl of the pAE expression vector (FIG. 4), 2 μl of buffer 5× for DNA ligase and 1 μl of DNA ligase. The clones, subcloned in pAE vector were also sequenced.

### Induction of the Lopap Expression

Aiming to obtain a great amount of soluble recombinant proteins, the BL21 (DE3) strain of the E. coli bacteria was preferably used for expressing this protein. It provides a fast growing, it is easy to be cultured and kept, as well as it presents a high quantity of recombinant proteins. This E. coli strain is lysogenic and does not present the post-translation modification systems.

E. coli cultures transformed preferably by the recombinant expression vector (pAE-clone 14.16) (FIG. 4) were inoculated in 3 ml of LB/ampicillin (100 μg/μl) medium and incubated at 37° C. until obtaining a DO_{600 nm} of 0.5. For the non-induced control, 1 ml of the pre-inoculum was maintained at 4° C. IPTG for 0.5 mM was added to the rest of the volume and the incubation was maintained for more 3 h. For every 40 μl of culture, 10 μl of SDS-PAGE application buffer with β-mercaptoethanol 0.1 M was added. The samples were boiled for 12 min and applied in polyacrylamide gel 12.5%. Afterwards, the gel was stained with 0.25% of “Coomassie Blue Brillant” in 50% methanol for 18 h and destained with acetic acid 10% in water for 3 h at room temperature.

For obtaining the expression of the protein (Lopap) of this invention, the following procedures were taken:

E. coli cultures transformed with the expression vector were inoculated in 100 ml of LB/ampicillin (100 μg/μl) medium and incubated at 37° C. until obtaining of a DO₆₀₀ nm of 0.5. Aliquots of 25 ml were incubated in 4 different bottles with 500 ml of LB/ampicillin (100 μg/μl) for 90 min. at 37° C. IPTG was then added for reaching the final concentration of 1 mM and the incubation was maintained for more 4 h. The medium was then centrifuged in 12000 rpm and frozen at −70° C. for 18 hrs. The cells of the 4 bottles were re-suspended in 70 ml of lysis buffer NaH₂PO₄ 50 mM, NaCl 300 mM, imidazol 10 mM and submitted to a French press of 2000 GAGE for three times and centrifuged in 5000 rpm for 15 min. at 4° C.

The supernatant containing the soluble express protein was centrifuged in 15000 rpm for 30 min for clarification and applied in nickel-sepharose affinity column previously balanced with lysis buffer. The column was washed with buffer imidazol 80 mM, β-mercaptoethanol 5 mM, NaCl 500 mM, Tris HCl 50 mM pH 6.8 and the washing volume was collected. The protein was eluded using Tris-HCl 50 mM pH 8.0, imidazol 1M, NaCl 100 mM with flow of 1 ml/5 min.

The “pellet” (corpuscles) of the medium was submitted to the French press and centrifuged, re-suspended in 20 ml of buffer Tris-HCl 50 mM, Urea 1 M, Triton X-100 1%, pH 8.0 for eliminating hydrophobic components and centrifuged in 5000 rpm for 15 min at 4° C. The separated precipitate was incubated at room temperature for 25° C. with 10 ml of buffer Tris-HCl 50 mM, NaCl 500 mM, Urea 8 M, β-mercaptoethanol 10 mM pH 8.0 for solubilization of the corpuscles. This material was again centrifuged in 4000 rpm for 20 min at 4° C. and the supernatant was added drop by drop into the “refolding” buffer of Tris-HCl 50 mM, NaCl 500 mM, Imidazol 5 mM and β-mercaptoethanol 5 mM pH 8.0 (as an alternative for obtaining the protein with the correct structure. Another approach for reaching this stage was performed using the buffer added with CaCl₂ 100 mM) with constant swirling at room temperature for 18 h. The material was filtrated and applied for 72 h in a nickel-sepharose column previously balanced with the lysis buffer. The column was washed with 180 ml of buffer Tris-HCl 50 mM, NaCl 500 mM, Imidazol 20 mM pH 6.8 and eluded with Tris-HCl 50 mM pH 8.0, imidazol 1M, NaCl 100 mM with a flow of 1 ml/5 min.

For isolating all the rLopap in its correct form, both the soluble protein and the eluded one provenient from the corpuscles were submitted to a benzamidine-sepharose column in medium of Tris-HCl 20 mM, NaCl 500 mM, pH 8.0 and eluded with glycine 50 mM, pH 3.0. The eluded protein was dialyzed exhaustively against NaCl 3 mM for 48 h.

The eluded protein as well as the aliquots of intermediate phases of the purification process were dosed by the Bradford method (1976) and analyzed by SDS-PAGE.

The recombinant protein obtained was tested concerning its prothrombin activating capacity using purified prothrombin and preferably the S-2238 chromogenic substrate (Chromogenix).

### The Tertiary Structure Model of Lopap

Based on the sequence obtained by cDNA, an approach of Lopap model study was conducted using the Swiss PDB Viewer 3.7 (b2) program and the Swiss Model Server.

### The Analysis of the Secondary Structure of the Recombinant Lopap

For evaluating the “folding” of the recombinant protein, its secondary structure was analyzed by Circular Dichroism spectrometry.

The spectrum (CD) was conducted in a spectropolarimeter at 25° C. between 190 and 300 nm wavelengths. The spectra were accumulated 8 times with a resolution of 1 nm in speed of 200 nm/min.

The recombinant Lopap was diluted in Tris/HCl 20 mM pH=8.0 buffer in a concentration of 1.2 mg/ml. The data were expressed as molar based on the protein concentration.

### EXAMPLE 1

### Preparing the mRNA

7.0 μg of mRNA was obtained, out of which 4 μg was used for the construction of a cDNA library.

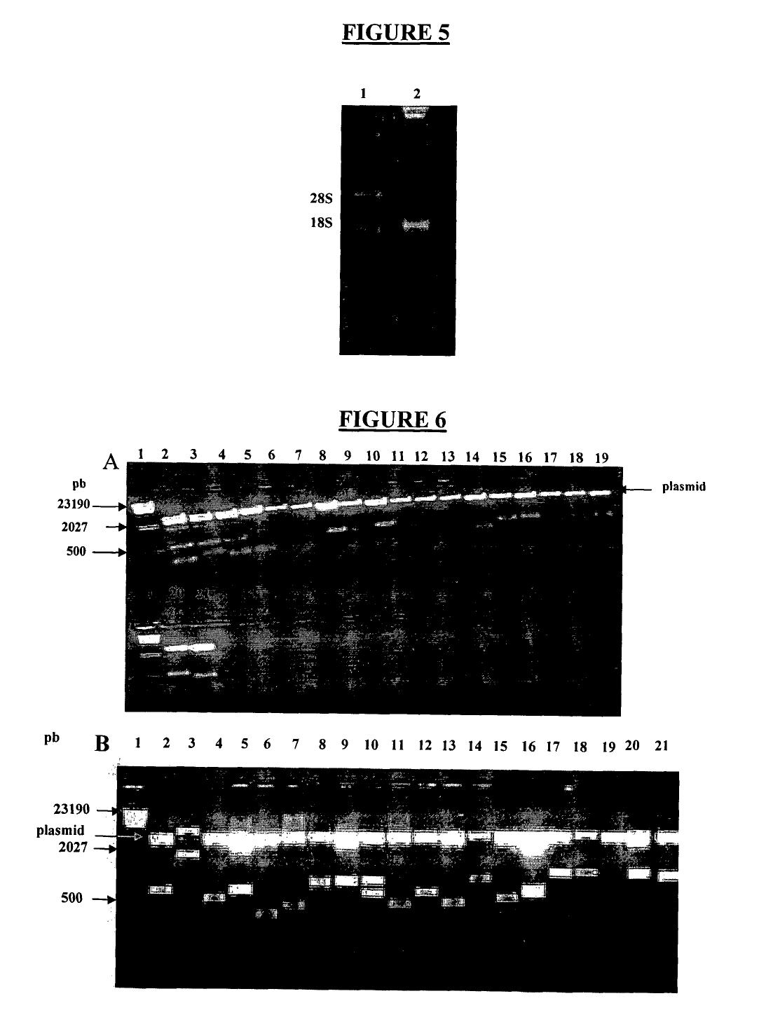

The mRNA obtained showed to have good quality (1.5:1 relation with proteins) and the analysis in agarose gel revealed a correspondent smir toward the mRNA (FIG. 5).

### EXAMPLE 2

### Constructing a Library of cDNA in Plasmids

Screening in agarose gel, the plasmid library presented a title of 10⁵ plasmids/μg. Two libraries were constructed in plasmid: one with inserts between 400 and 800 pb (BA) and the other with inserts larger than 800 pb (BB).

From each library 300 clones were randomly selected, that after digestion with EcoR I and Hind III presented a variety of inserts of different sizes. Some of these inserts were digested by the restriction enzymes, producing two fragments in agarose gel (FIG. 6). 300 clones randomly selected in the cDNA BA and BB libraries were sequenced and presented significant identity with the proteins available in the “GenBank”.

### EXAMPLE 3

### Amplification of the Lopap Codifying cDNA

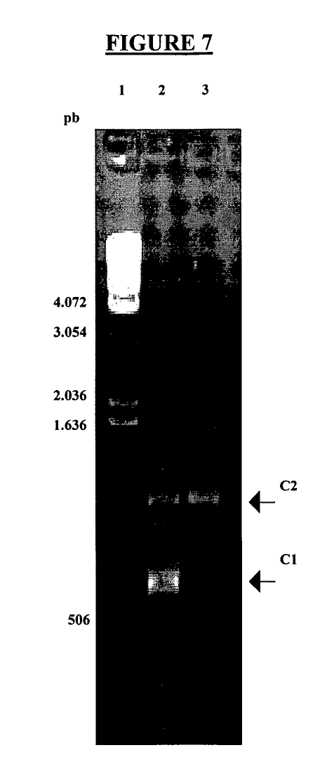

The product amplifying with the oligonucleotides P1 and SP6 of the BA library (400 to 800 pb) showed a band of approximately 600 pb and another of around 800 pb, while the BB library (larger than 800 pb) showed a band of around 800 pb (FIG. 7). The 600 pb band (named C1) and the 800 pb band (named C2) were cut out of the gel and purified. The purified cDNA was subcloned preferably in “easy” pGEM-T and the DH5α competent bacteria were transformed and placed in plates.

### EXAMPLE 4

### Screening the Recombinant Plasmids

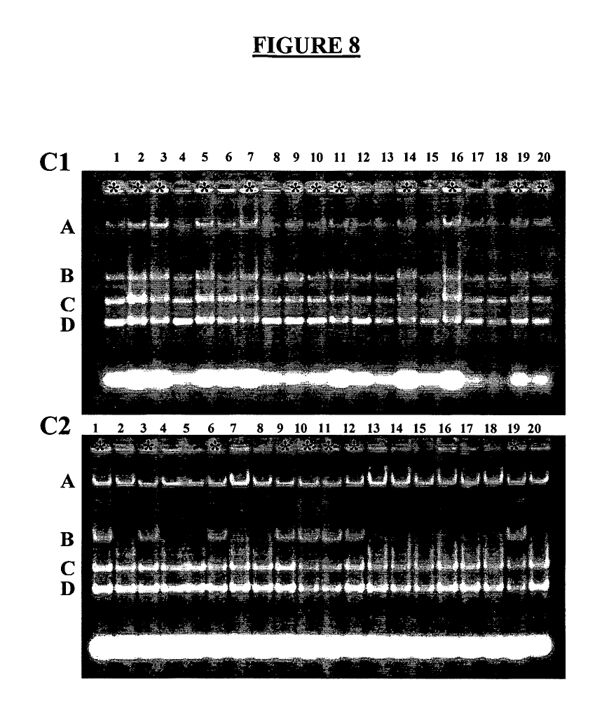

40 clones were collected (example 3), 20 referred to the C1 band (named from C1-1 to C1-20) and the other 20 clones referred to the C2 band (from C2-1 to C2-20), and submitted to the screening process concerning the size of the insert, before the plasmidial DNA purifying through the mini-preps. As demonstrated in FIG. 8, the plasmids presenting large inserts were purified and submitted to the PCR essays using the primer P2.

### EXAMPLE 5

### Amplifying by PCR with the Primer P2

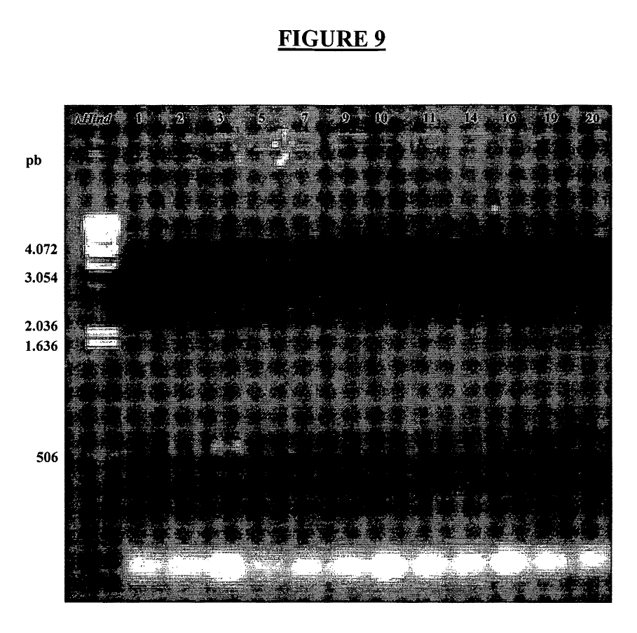

The positive clones from the previous item were amplified with P2 and SP6 and only the clones 2 and 3 of C1 (C1-2 and C-1-3) were positive (FIG. 9) and were submitted to the restriction and sequencing essays. The clones referring to the C2 band were not shown for being all negative clones.

### EXAMPLE 6

### Digestion of Plasmidial DNAs with Restriction Enzymes

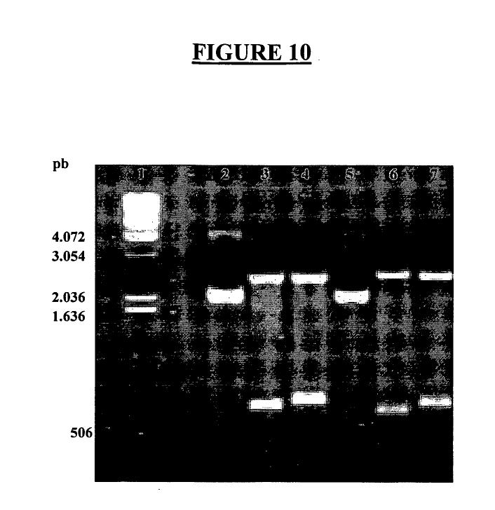

The DNAs C1-2 and C1-3 subcloned in “easy” pGEM-T were aligned after the cleavage of the BamH I site and Hind III and also after the cleavage of the EcoR I sites liberating inserts with around 600 pb (FIG. 10).

### EXAMPLE 7

### cDNA Sequencing

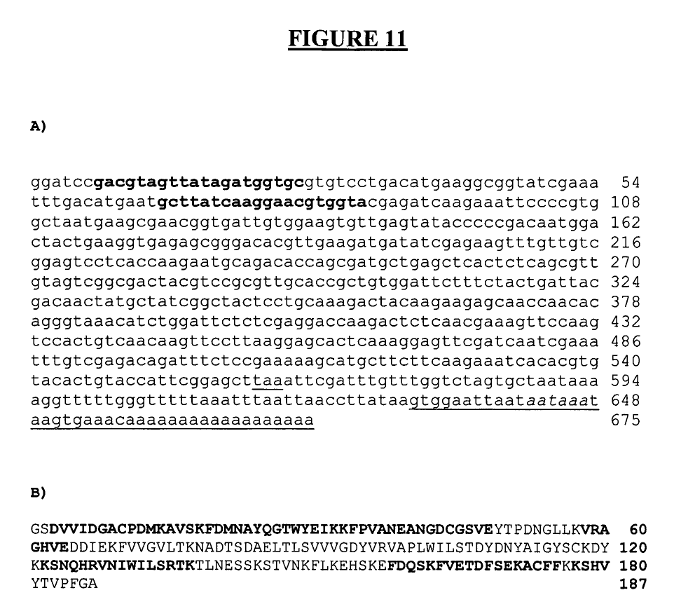

The clones of the PCR product of 600 pb (C1-2 and C1-3), as well as the clones bound to the pAE expression vector with the same cleavage profile, were sequenced and contained the sequence of the initiator oligonucleotides P1 and P2 and the sequences referring to the 46 residues of the Lopap N-terminus, as well as of the internal fragments previously sequenced. 561 nucleotides were sequenced corresponding to 187 amino acids of the total sequence of the protein (FIG. 11).

### EXAMPLE 8

### Expression

The insert liberated from the “easy” pGEM-T with the restriction enzymes of BamH I and EcoR I was subcloned in pAE vector with reading phase with the end of the six histidines of this vector.

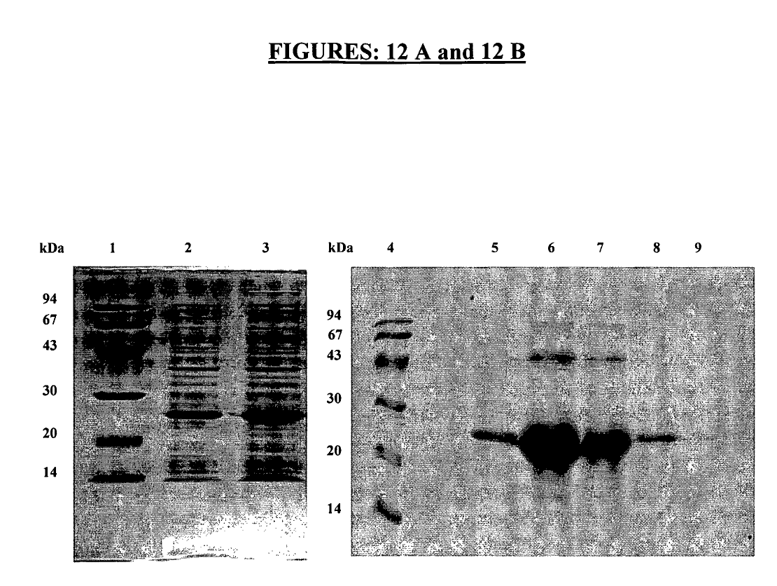

The recombinant protein with 21 kDa, including the 6 histidine residues added by the pAE vector, was expressed before the induction with IPTG and its production was increased by induction (FIG. 12A). Although soluble expressed protein was found, the major part of Lopap was composed of inclusion corpuscle that, after solubilization with urea and β-mercaptoethanol and its purification (FIG. 12B) in nickel-sepharose column, it was submitted to a benzamidine-sepharose column and eluded by pH alteration for isolating the protein in its correct structure.

The rLopap was dialyzed against EDTA 3 mM and its prothrombin activating activity was tested using S-2238 chromogenic substrate and also in presence of platelets.

### EXAMPLE 9

### Analyzing the Lopap Sequence

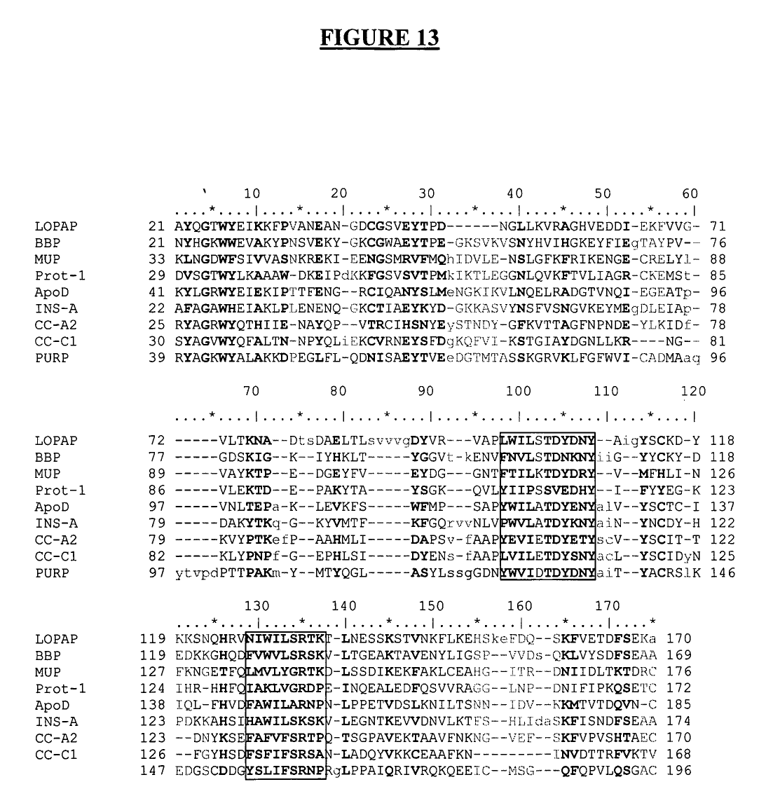

The sequence deduced for Lopap showed an identity of around 30% with several proteins of the lipocalin family (FIG. 13). A high similarity in the areas responsible for the tertiary structure, characteristics of the lipocalins, placed around the Leu118-Tyr128 residues and the Asn149-Lys157 residues.

### EXAMPLE 10

### Three-Dimensional Structure Model of Lopap

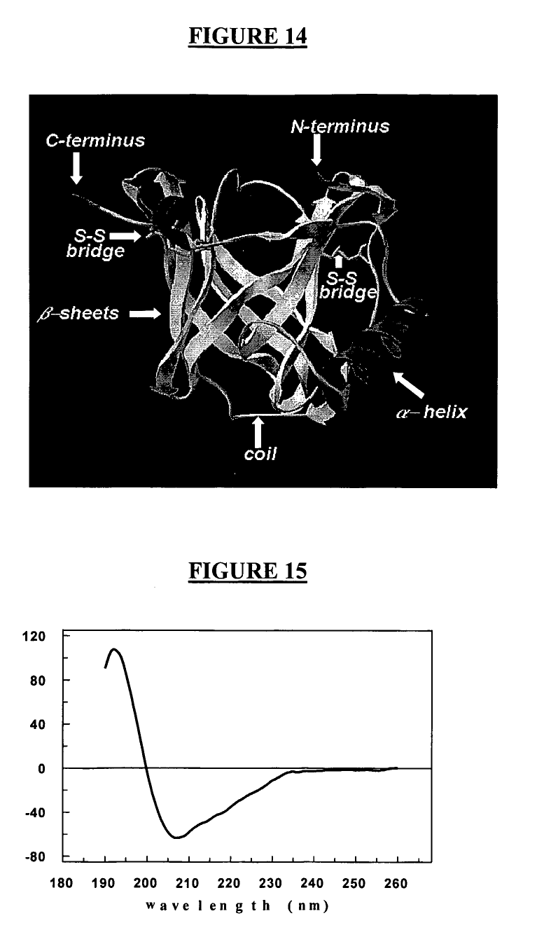

The three-dimensional structure of Lopap, analyzed for modeling and compared to the data banks, showed similarities to a structure quite characteristic to those of the lipocalin family proteins (FIG. 14). It includes 9 segments of β-sheet kind, a β-barrel region and two α-helices, one placed at the N-terminus and the other at the C-terminus area. Besides that, the possibility of forming two intramolecular disulphide bridges could also be observed.

### EXAMPLE 11

### Study of the Recombinant Lopap Secondary Structure by Circular Dichroism

Peptides and proteins present a standard spectrum of secondary structure that can be evaluated by Circular Dichroism spectrometry (CD). The most significant and characteristic standard is represented by two α-helices (Holzawarth et al, 1965), in which we can see two negative bands of comparable magnitude close to 222 and 208 nm and one positive band close to 190 nm.

The spectrum presented by the β-sheet is of lower intensity, showing a negative band close to 217 nm, a positive one close to 195 nm, and another negative band close to 180 nm (Brahms et al, 1977).

The lipocalins have characteristic structural elements showing approximately 7% of α-helices, 47% β-sheet and 45% randomized structures. (Flower, 1996). The circular dichroism spectrum of Lopap (FIG. 15) showed characteristics that are typical of the lipocalin family with 6.2% of α-helices, 52.9% of β-sheets and 28.9% randomized structures.

### EXAMPLE 12

### Prothrombin Hydrolysis

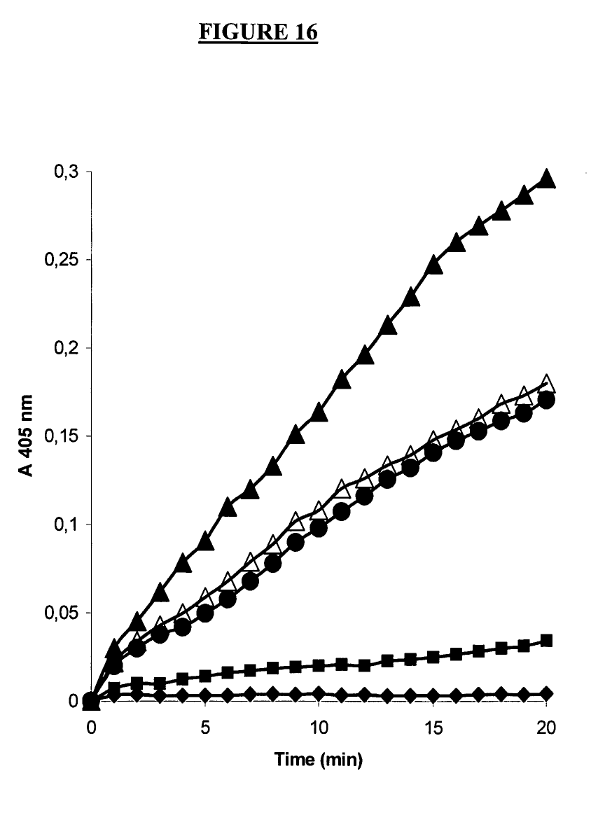

The native Lopap and the rLopap are able to activate prothrombin using preferentially the S-2238 substrate. However, in same concentrations, rLopap is less efficient (FIG. 16). None of the two proteins is able to submit hydrolysis directly in chromogenic substrate.

### EXAMPLE 13

### Prothrombin Activation by rLOPAP

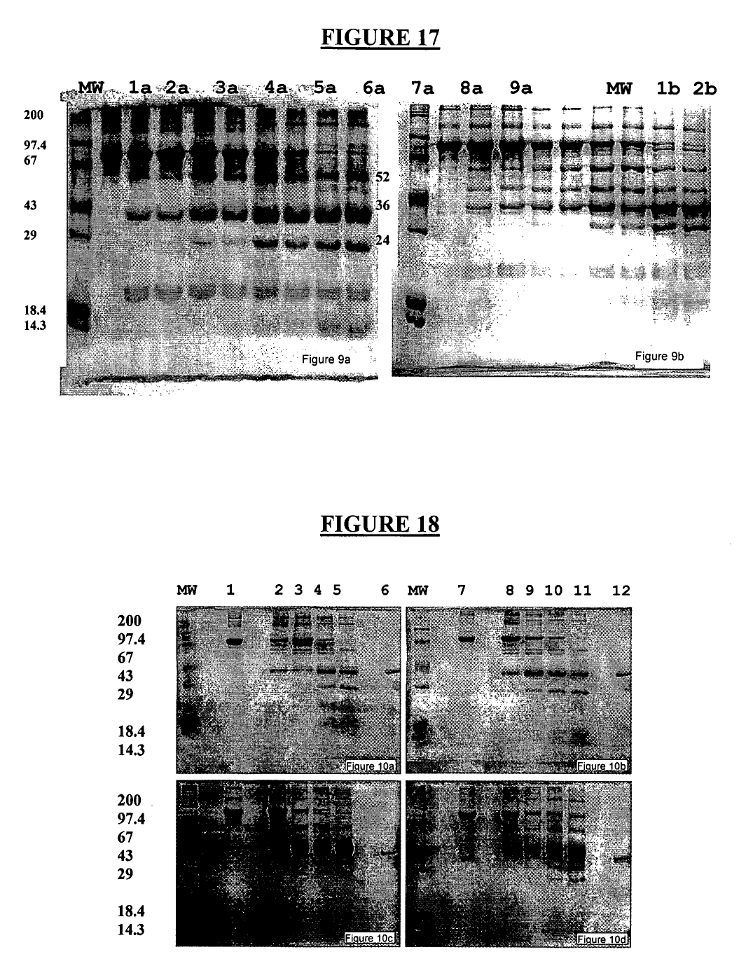

The prothrombin (10 μM) was activated by rLOPAP (2 μM) in presence and absence of 5 μM phospholipids (phosphatidyl Serine:phosphatidyl Colina PS:PC), in reaction buffer (Tris-HCl 0.02M, NaCl 0.15M pH 8.0 with CaCl₂15 mM). Aliquots of 10 μl were collected in different times of the reaction (1 min; 1 h 30 m; 5 h and 18 h) for being analyzed in SDS-PAGEs 10% (FIG. 17).

Under non-reduced conditions bands of 72 kDa (prothrombin), of 52 kDa probably corresponding to the prothrombin F1.2, one of 36 kDa corresponding to the thrombin or to the prethrombin 2, and one of 24 kDa corresponding to the fragment 1 (F1) of the prothrombin could be observed. Under reduced conditions, bands of 52 kDa (F1.2), 36 kDa (prethrombin 2), 32 (B chain of the prothrombin) and 27 kDa (F1) were observed.

For verifying the influence of the prothrombinase complex components in the prothrombin activation by r-LOPAP, the prothrombin (10 μM) was activated by r-LOPAP (2 μM) with PS:PC 5 mM in presence and in absence of the Va 200 μM Factor. The reaction buffer used was Tris-HCl 0.02M, NaCl 0.15M pH 8.0 with CaCl₂ 15 mM. The aliquots (10 μl) were collected in different times of incubation (1 min; 1 h 30 m; 5 h and 18 h) for being analyzed by SDS-PAGE (FIG. 18). The same hydrolysis standards were obtained in presence and in absence of the Factor Va.

### EXAMPLE 14

### In Vivo Studies of the Defibrinating Capacity of rLopap

Mice treated with rLopap intravenous administrations of 125, 250 and 500 μg/kg, considering the weight of the animal, were maintained alive and showed good life conditions after 48 hours of treatment. However blood showed unclotability and the plasmatic fibrinogen levels measured by the Clauss test were undetectable either after 2 and 48 hours after the rLopap administration (Table 1). Doses of 500 μg/kg did not show any toxic effect in the animals that continued without clear alterations, only presenting a longer time of coagulation period that lasted for at least 48 h.

Treatment with doses lower than 125 μg/kg can turn longer the coagulation time of the fibrinogen without provoking severe alterations in the microcirculation.

TABLE 1
Dosage of Fibrinogen in the plasma of mice treated with rLopap

| | Time of fibrin clot | Time of fibrin clot |
|---|---|---|
| rLopap dose | formation after 2 h | formation after 48 h |
| μg/kg | of treatment | of treatment |
| Control | 38 s | 41 s |
| 125 | Unclotable | — |
| 250 | Unclotable | Unclotable |
| 500 | Unclotable | Unclotable |

### EXAMPLE 15

### Prothrombin Activator Activity

The capacity of the recombinant form of native Lopap, (rLopap) in activating prothrombin was indirectly determined through the thrombin formation essay when considering the prothrombin with the S-2238 chromogenic substrate. The prothrombin activator activity of the protein (15 nM) was evaluated after pre-incubation for 20 min at 37° C. with prothrombin (90 nM), in presence of CaCl₂ 5 mM for a final volume of 100 μl. This reaction occurred in Tris-HCl 50 mM, NaCl 100 mM, pH 8, 3, containing imidazol 150 mM. The hydrolysis of S-2238 40 μM by the thrombin formed by Lopap, using 90 nM of prothrombin was followed spectrophotometrically in 405 nm for 20 minutes at 37° C.

### EXAMPLE 16

Aiming to verify whether rLopap can be used as a component of a diagnostic kit for prothrombin dosage, 50 μl of human plasma was diluted 1/40 in buffer TRIS-HCl 20 mM, NaCl 100 mM, pH 8.0, incubated 5 min at 37° C. with 15 μl of CaCl₂ 50 mM and 40 μl of rLopap for final concentration of 5 μg/ml. After that, 20 μl of S2238 substrate (Chromogenix) 3 mM was added to that and incubation was conducted for more 5 min at 37° C. The reaction was interrupted with 50 μl of acetic acid 30% and the substrate hydrolysis was measured spectrophotometrically in 405 nm.

The prothrombin concentration was calculated in accordance with a standard curve obtained from the dilutions of 1/30, 1/40, 1/80 and 1/160 (150, 100, 50 and 25% of activity respectively) of standard human plasma (Dade-Behring) prepared using the same procedures.

The prothrombin concentration in a human plasma sample showed 93% of activity. Controls available in the market (normal and pathologic-Dade-Behring) were used for validating this result. For the normal control (70-100% of activity) 86% was obtained as result and for the pathologic control (35%-50%) 41% of activity was reached. The prothrombin deficient plasma, used as control, showed a prothrombin concentration of 5%. These data indicate that rLopap can be used for determining prothrombin levels in plasmas of patient.

FIG. 1. Degenerated primers for the amplification of the clone corresponding to Lopap. A=adenine; C=cytosine; T=thymine; G=guanine; M=C or A Y=T or C R=A or G N=A, T, C or G

FIG. 2. Map of the pGEM-11Zf(+) (Promega-TBO75)

FIG. 3. Map of the “easy” pGEM-T (Promega-A1360)

FIG. 4. Map of the pAE (Biotechnology Center-Butantan Institute-Ramos et. al., 2003)

FIG. 5. Eletrophoretic profile agarose gel 1% of the mRNA. 1: degraded RNA used as comparative control; 2: mRNA extracted from the bristles.

FIG. 6. cDNA inserts of the plasmid library. Electrophoresis in agarose gel 1% showing the fragments produced by 40 clones randomly selected from the plasmid library, after the incubation for 2 hours with EcoR I and Hind III. (A) cDNA library ranging from 400 to 800 pb (BA) and (B) cDNA library with inserts larger than 800 pb (BB). 1: HindIII Marker; 2 to 21: cleavage products of the aleatory clones

FIG. 7. Product of the cDNA amplification that codifies Lopap. Agarose gel 1%. 1: Lambda/Hind III marker; 2: Product amplified with the oligonucleotides P1 and SP6 of BA; 3: Product amplified with the oligonucleotides P1 and SP6 of BB. C1 and C2 are the amplified fragments of 600 and 800 pb.

FIG. 8: Screening recombinant plasmids by size in agarose gel 1%. (A) Genomic DNA; (B) recombinant plasmids with large inserts; (C) plasmids empty or with small inserts; (D) RNA.

Positive clones with inserts amplified by primer 1.

FIG. 9. Inserts amplification using primer P2. Positive clones referent to band C1, previously amplified with P1. Only the clones 2 and 3 were positive.

FIG. 10: Eletrophoretic profile of the PCR 1 product cleavage: Lambda/Hind III marker; 2: plasmidial DNA of C1-2; 3: vector (3.000 pb) and insert liberated (600 pb) after cleavage of C1-2 with BanHI and Hind III; 4: vector and insert liberation after cleavage of C1-2 with EcoR I; 5: plasmidial DNA of C1-3; 6: vector and insert liberated after cleavage of C1-3 with BanH I and Hind III; 7: vector and insert liberated after cleavage of C1-3 with EcoR I

FIG. 11: Nucleotide sequence obtained for the rLopap and deduced of amino acids. A) Nucleotide sequence, SEQ ID No.1: In bold we see the sequences of the oligonucleotides P1 (7 to 29) and P2 (67 to 86) and the stop codon (562 to 564) and the polyadenylation site (630 and so on) underlined. B) Amino acids sequence SEQ ID No.2: In bold we see the sequences of amino acids obtained by chemical sequencing. The two first amino acids are referent to the BamHI site added to the primers.

FIG. 12. Lopap expression. The recombinant protein, produced in E. coli, after induction with IPTG was purified in nickel-sepharose column. 1 and 4: Standard; (A)rLopap expression-2: Expression before adding IPTG; 3: Expression after adding IPTG; and (B)rLopap purification; 5 a 9: fractions eluded in the purification

FIG. 13. Alignment of Lopap deduced sequences of amino acids compared with other members of the lipocalin family.

The sequences were obtained in the “GenBank” data bank, showing similarities between Lopap and the proteins of the lipocalin family. (pfam00061) with Score=49.3 bits and E-value 2e-07. BBP—BILIN-BINDING PROTEIN, Pieris brassicae (gi 1705433); MUP—MAIN PRECURSOR OF THE URINARY PROTEIN, Rattus norvegicus (gi 127533); Prot-1—PROTEIN 1 OF THE VON EBNER'S GLAND, Rattus norvegicus (gi 12621114); ApoD PRECURSOR OF THE APOLIPOPROTEIN D, Homo sapiens (gi 4502163); INS-A —INSECTICYANIN A FORM Manduca sexta (gi 124151); CC-A2—SUB-UNITY A2 OF THE CRUSTACYANIN A2 Homarus gammarus (gi 117330); CC-C1.—SUB-UNITY C1 OF THE CRUSTACYANIN Homarus gammarus (gi 117420); PURP.—PRECURSOR OF THE PURPURIN Gallus gallus (gi 131549). High similarity represented in bold. The regions with high similarities with lipocalin characteristics are emphasized.

FIG. 14. Three-dimensional structure of Lopap. Structure model using the Swiss PDB Viewer 3.7 (b2) and Swiss Model Server programs.

FIG. 15. Spectrum of rLopap circular dichroism. The spectrum (CD) was conducted in a spectropolarimeter at 25° C. between 190 and 300 nm wavelengths. Spectra accumulated 8 times with resolution of 1 nm in a speed of 200 nm/min.

FIG. 16. Prothrombin Activation. 5 μg of activator incubated with 90 nM of prothrombin for 100 μl of final volume at 37° C.

substrate in absence of FII and activators (white); ▪ FII control without activator; rLopap and Δ native Lopap.

FIG. 17. Prothrombin hydrolysis by rLopap. SDS-PAGE in 10% stained by Coomassie Blue under non-reduced conditions (FIG. 17a) and reduced (FIG. 17b).

MW Marker [Myosin (200 Kda), Phosphorylase B (97.4 KDa), BSA (67 KDa), Ovalbumine (43 KDa), Carbonic Anidrase (29 KDa), b-Lactoglobulin (18.4 KDa), Lysozyme (14.3 KDa); Prothrombin (PT);
1) PT+LOPAP (1 min inc); 2) PT+LOPAP+PS:PC (1 min inc); 3) PT+LOPAP (1 h 30 min inc); 4) PT+LOPAP+PS:PC (1 h 30 min inc); 5) PT+LOPAP (5 h inc); 6) PT+LOPAP+PS:PC (5 h inc); 7) PT+LOPAP (18 h inc); 8) PT+LOPAP+PS:PC (18 h inc)

FIG. 18. Prothrombin Hydrolysis in presence and absence of the prothrombinase complex factors. SDS-PAGE stained by Coomassie Blue. Non-reduced conditions (FIGS. 18a and 18c) and reduced conditions (FIGS. 18b and 18d).

MW Marker [Myosin (200 Kda), Phosphorylase B (97.4 KDa), BSA (67 KDa), Ovalbumine (43 KDa), Carbonic anidrase (29 KDa), b-Lactoglobulin (18.4 KDa), Lysosima (14.3 KDa);
1) Prothrombin (PT); 2) PT+LOPAP+PS:PC+Va (1 min inc); 3) PT+LOPAP+PS:PC+Va (90 min inc); 4) PT+LOPAP+PS:PC+Va (5 h inc); 5) PT+LOPAP+PS:PC+Va (18 h inc); 6) Thrombin, 7) Prothrombin (PT); 8) PT+LOPAP+PS:PC (1 min inc); 9) PT+LOPAP+PS:PC (90 min inc); 10) PT+LOPAP+PS:PC (5 h inc); 11) PT+LOPAP+PS:PC (18 h inc); 12) Thrombin

### REFERENCES

Beuken E; Vink C; Bruggeman C A. One-step procedure for screening recombinant plasmids by size. Biotechniques 1998; 24:748-50.
Bradford MM. A rapid and sensitive method for the quantities of protein utilizing the principle of protein dye binding. Analytical Biochem 1976; 72:248-254.
Brahms S; Brahms J; Spach G; Brack A. Identification of beta, beta-turns and unordered conformations in polypeptide by vacuum ultraviolet circular dichroism. A. Proc. Natl. Acad. Sci. 1977; 74: 3208.
Brasil. Ministério da Saúde. Fundação Nacional de Saúde.-Acidentes por lepdópteros. In: Manual de diagnóstico e tratamento de acidentes por animais peçonhentos. Brasília, 1998. 131 p.
Chen BP; Twons, H. Expression vectors for affinity purification and radiolabeling of proteins using Escherichia coli as host. Gene 1994; 139: 73-75,
Chudzinski-Tavassi A M, Schattner M, Fritzen M, Pozner R G, Reis C V, Lourenço D, Lazzari M A. Effects of Lopap on Human Endothelial Cells and Platelets. Haemostasis 2001; 31:257-265.
Donato J L; Moreno R A; Hyslop S; Duarte A; Antunes E; Le Bonniec B F, Rendu F, Nucci G. Lonomia obliqua Caterpillar Spicules Trigger Human Blood Coagulation via Activation of Factor X and Prothrombin. Thromb. Haemost 1998; 79: 539-42.
Duarte A C; Caovilla J; Lorini I; Lorini D; Mantovani G; Sumida J; Manfre P C; Silveira R C; Moura S P. Insuficiência renal aguda por acidentes com lagartas. J Bras Nefrol 1990; 12(4):184-187.
Flower, D R; North, A C T, Sansom, C E. The lipocalin protein family: structural and sequence overview. Biochim Biophys Acta 1482: 9-24, 2000.
Holzawarth G; Dotv P. J. Am. Chem. Soc. 1965; 87: 218.
Inoue H; Hojima H; Okayama H.-High efficiency transformation of E. coli with plasmids. Gene 1990; 96: 23-28.
Kelen, E M A; Picarelli, Z P; Duarte, A C.-Hemorrhagic syndrome induced by contact with caterpillars of the genus Lonomia (Saturniidae, Hemoleucinae). J. Toxicol-Toxin Rev., 1995; 14:283-308.
Lemaire C. Revision du genre Lonomia Walker (Lep Attacidae). Ann Soc Entomol Fr 1972:8:767-861.
Lorini, L M.-A taturana: aspectos biológicos e morfológicos da Lonomia obliqua. Passo Fundo: EDIUPF, 1999. p. 25-35.
Prezoto B C; Maffei F H A; Mattar L; Chudzinski-Tavassi A M; Curi P R. Antithrombotic effect of Lonomia obliqua caterpillar bristle extract on experimental venous thrombosis. Braz J Med Biol Res 2002; 35(6): 703-12.
Ramos C R; Figueredo R C; Pertinhez T A; Vilar M M; do Nascimento A L; Tendler M; Raw I; Spisni A; Ho P L. Gene structure and M20T polymorphism of the Schistosoma mansoni Sm14 fatty acid-binding protein. Molecular, functional, and immunoprotection analysis. J Biol Chem 2003; 278(15):12745-51.
Reis C V; Farsky S H P; Fernandes B L, Santoro M L; Oliva M L V; Mariano M and Chudzinski-Tavassi A M. In Vivo Characterization of Lopap, a Prothrombin Activator Serine Protease from the Lonomia obliqua Caterpillar Venom. Thromb. Res 2001; 102: 437-43.
Reis C V; Kelen E M A; Farsky S H P; Portaro F C V; Sampaio C A M; Fernandes B L; Camargo A C M; Chudzinski-Tavassi A M. A Ca⁺⁺ activated serine protease (Lopap) could be responsible for the haemorragic syndrome caused by the caterpillar Lonomia obliqua. Lancet 1999; 353: 1942.
Reis C V; Portaro F C V; Andrade A S; Fritzen M; Fernande B; Sampaio C A M; Camargo A C M; Chudzinski-Tavassi A M. A prothrombin activator serine protease from the Lonomia obliqua caterpillar venom (Lopap): biochemical characterization Thromb Res 2001; 102:427-436.
Sambrook J; Fritsch E F; Maniatis T.-Molecular cloning, a laboratory manual, 2nd ed., Cold Spring Harbor, N Y, Cold Springer Harbor Laboratory press, 1989.
Zannin M; Lourenço DM; Motta G; Costa L R D; Granda M; Gaboje G; Noguti M A; Chudzinski A M. Blood coagulation and fibrinolytic factors in 105 patients with hemorrhagic syndrome caused by Accidental contact with Lonomia obliqua caterpillar in Santa Catarina, southern Brazil. Thromb Haemost

1.-65. (canceled)

66. A NUCLEOTIDE SEQUENCE and functional equivalent sequences thereof ENCODING FOR A RECOMBINANT PROTHROMBIN ACTIVATOR PROTEASE (rLOPAP) comprising SEQ ID NO. 1.

67. The NUCLEOTIDE SEQUENCE according to claim 66, comprising sequences of the oligonucleotides P1 (7 to 29) and P2 (67 to 86), stop codon (562 to 564) and the polyadenylation site (630 and up).

68. AN ACTIVATOR SEQUENCE OF RECOMBINANT PROTHROMBIN (rLOPAP) comprising SEQ ID NO: 2.

69. The ACTIVATOR SEQUENCE according to claim 68, wherein the two first amino acid residues correspond to the restriction site.

70. The ACTIVATOR SEQUENCE according to claim 68, having a sequence identity of around 30% as compared with proteins of the lipocalin family.

71. The ACTIVATOR SEQUENCE according to claim 68, having a substantial similarity concerning lipocalin tertiary structure regions.

72. A RECOMBINANT PROTHROMBIN ACTIVATOR PROTEASE (rLOPAP) having:
(a) in its monomer form, a molecular weight of approximately 21 kDa;
(b) a secondary structure defined by the circular dichroism (CD) spectrum as represented in FIG. 15 as measured in a spectropolarimeter at 25° C. and at wave lengths ranging from 190 and 300 nm;
regions responsible for its tertiary structure as represented in FIG. 14; and
(d) a sequence identity of around 30% as compared with proteins of the lipocalin family.

73. The RECOMBINANT PROTHROMBIN ACTIVATOR PROTEASE, according to claim 72 wherein it is encoded by a nucleotide sequence as set forth in SEQ ID NO: 1.

74. The RECOMBINANT PROTHROMBIN ACTIVATOR PROTEASE, according to claim 72 having an amino acid sequence as forth in SEQ ID NO: 2.

75. The RECOMBINANT PROTHROMBIN ACTIVATOR PROTEASE, according to claim 72, wherein the CD spectra is accumulated 8 times with a resolution of 1 nm at a speed of 200 nm/min.

76. A PROCESS FOR OBTAINING A RECOMBINANT PROTHROMBIN ACTIVATOR PROTEASE (rLOPAP) IN MONOMERIC FORM comprising:
(a) obtaining the mRNA from bristles of the Lonomia obliqua caterpillar;
(b) forming a cDNA library from the mRNA of step (a);
(c) binding the cDNA to the adapters and to a cloning vector containing the site of transcription initiation of the T7 RNA polymerase and the operon Lac Z initiating sequence;
(d) transforming the resulting cDNA in appropriate prokaryote cells;
(e) amplifying the cDNA resulting from step (d) that encodes for the desired prothrombin activator protein;
(f) binding the DNA resulting from step (e) to a subcloning vector containing the sequence of the ampicillin-resisting gene and screening the required recombinant plasmids;
(g) amplifying the cDNA obtained from step (f), digesting the resulting plasmid DNA with restriction enzymes; and
(h) carrying out the resulting DNA sequencing;
(i) expressing the recombinant protein rLOPAP.

77. The PROCESS according to claim 76, wherein the mRNA of step (a) is obtained by:
(i) withdrawing the spicules of L. obliqua and freezing them in very low temperatures;
(ii) grinding the spicules treated for eliminating the RNAse, and adding to the resulting fine powder with a sufficient quantity of solution of phenol and guanidine isothiocyanate; and
(iii) extracting the resulting RNA.

78. The PROCESS according to claim 77 characterized by further purifying the extracted mRNA.

79. The PROCESS according to claim 76 wherein the cDNA library of step (b) is obtained by:
(i) diluting the mRNA of step (a), heating it up at 70° C. for 10 minutes, cooling it in ice bath and centrifuging it rapidly.
(ii) obtaining the first cDNA strain from the mRNA of step (ii); and
(iii) further obtaining the second cDNA strain.

80. The PROCESS according to claim 79, wherein the cDNA is obtained by the RT-PCR technique.

81. The PROCESS according to claim 79, wherein the resulting cDNAs have fragments length ranging from 400 to 800 pb in the low molecular weight (BA) band, and fragments length over 800 pb in the high molecular weight (BB) band.

82. The PROCESS according to claim 79, wherein the first fragment size screening is carried out in agarose gel.

83. The PROCESS according to claim 79, wherein the fragments of high and low molecular weight are applied in agarose gel, and after electrophoresis the bands are cut out of the gel and the cDNAs of high and low weights are purified and eluded.

84. The PROCESS according to claim 83 wherein the cDNA is heated in a temperature ranging from 60 to 70° C., and concentrated under vacuum.

85. The PROCESS according to claim 76 wherein EcoRI adapters are used.

86. The PROCESS according to claim 76 wherein the binding of the cDNA to the cloning vector is unidirectional and occurs at 16° C. during 18 hours.

87. The PROCESS according to claim 86 wherein the cloning vector is preferentially digested with the EcoRI and NOTI enzymes.

88. The PROCESS according to claim 76 wherein the prokaryote cells are E. coli cells.

89. The PROCESS according to claim 76 wherein the transforming step (d) is carried out by the Inoue method.

90. The PROCESS according to claim 89 wherein the screening of the transformed prokaryote cells is carried out by using antibiotic.

91. The PROCESS according to claim 90, wherein the antibiotic is ampicillin.

92. The PROCESS according to claim 76 wherein the library amplifying step (d) is carried out by:
(i) incubating mixtures of DH5 competent bacteria and cloning vector bound to the DNA of high or low molecular weight during from 25 to 35 min in ice, from 2 to 3 min at 40° C., at 45° C. and again in ice for more 5 min.
(ii) adding a 2YT medium containing ampicillin in concentration ranging from 10 to 200 g/ml and homogenizing the resulting solutions.
(iii) taking aliquots in deaerated tubes and incubating at 37° C. from 16 to 20 h.
(iv) extracting the plasmid DNA.

93. The PROCESS according to claim 76 wherein PCR technique is used for amplifying the cDNA which encodes for the prothrombin activator protein.

94. The PROCESS according to claim 93 wherein 30 amplification cycles with a denaturation temperature of 94° C. are carried out, and annealing temperature of 50° C. and an extension temperature of 72° C. for DNA amplification.

95. The PROCESS according to claim 76 wherein available cloning vector and subcloning vector are used, as well as an expression vector which displays a marker for protein purification.

96. The PROCESS according to claim 95 wherein the cloning vector is a pGEM11zf(+) vector.

97. The PROCESS according to claim 95 wherein the subcloning vector is a “easy” pGEM-T vector.

98. The PROCESS according to claim 95 characterized by a DNA releasing of said cloning vector which is further extracted and purified.

99. The PROCESS according to claim 95 wherein the binding reaction of the DNA which encodes for the rLopap to the subcloning vector is carried out in an E coli DH5α system.

100. The PROCESS according to claim 76 wherein the expression vector is a pAE.

101. The PROCESS according to claim 76 wherein an automatic sequencing process for DNA sequencing is applied.

102. The PROCESS according to claim 101 wherein the initiator oligonucleotides “sense” P1 and P2 obtained from the protein N-terminal sequence are applied.

103. The PROCESS according to claim 101 wherein the oligonucleotides “sense” T7 and “anti-sense” SP6 are used.

104. The PROCESS according to claim 76 wherein the protein expression is carried out in prokaryote cells.

105. The PROCESS according to claim 104 wherein a BL21(DE3) strain is applied for obtaining high performance of the recombinant protein and great amounts of soluble recombinant protein.

106. The PROCESS according to claim 104 wherein a lysogenic strain for expressing the recombinant protein is used.

107. The PROCESS according to claim 76 wherein the recombinant protein is purified by using chromatographic techniques.

108. The PROCESS according to claim 107, comprising:
chromatographic columns of nickel-sepharose and/or benzamidine-sepharose; and
a nickel-sepharose column which is balanced with NaH2PO4 50 mM, NaCl 300 mM, imidazol 10 mM and eluded in buffer Tris-HCl 50 mM pH 7.0 in 9.0, imidazol 1M, NaCl 100 mM.

109. The PROCESS according to claim 108 wherein the elution buffer contains glycine.

110. The PROCESS according to claim 108 wherein the rLopap is obtained with a correct or a denatured structure.

111. The PROCESS according to claim 108 wherein the purified protein is dosed by the Bradford method and analyzed by SDS-PAGE.

112. The PROCESS according to claim 107 wherein the benzamidine-sepharose column is balanced in basic pH and eluded in acid pH.

113. The PROCESS according to claim 112 wherein a basic pH ranging from 7.5 to 9.0 and an acid pH ranging from 2.0 to 4.0 is used.

114. A RECOMBINANT PROTHROMBIN ACTIVATOR PROTEASE (rLOPAP) wherein it is obtained according to the process of claim 76.

115. The RECOMBINANT PROTHROMBIN ACTIVATOR PROTEASE, according to claim 114, wherein it is diluted in Tris/HCl buffer pH ranging from 7.8 to 8.5 and the data are expressed in a molar concentration basis.

116. USE of the recombinant prothrombin activator protease (rLOPAP) of claim 72 in a prothrombin diagnostic kit for analyzing plasma of patients with hemorrhagic problems.

117. USE of the recombinant prothrombin activator protease (rLOPAP) as obtained according to the process of claim 76, in a prothrombin diagnostic kit for analyzing plasma of patients with hemorrhagic problems.

118. USE of the recombinant prothrombin activator protease (rLOPAP) of claim 72 as a plasmatic fibrinogen consuming or depleting protein.

119. USE of the recombinant prothrombin activator protease (rLOPAP) as obtained according to the process of claim 76 as a plasmatic fibrinogen consuming or depleting protein.

120. USE of the recombinant prothrombin activator protease (rLOPAP) of claim 72 as a prothrombin activator.

121. USE of the recombinant prothrombin activator protease (rLOPAP) as obtained according to the process of claim 76 as a prothrombin activator.

122. USE of the recombinant prothrombin activator protease (rLOPAP) of claim 72 in clinical Pharmacology essays.

123. USE of the recombinant prothrombin activator protease (rLOPAP) obtained according to the process of claim 76 in clinical Pharmacology essays.

124. USE of the recombinant prothrombin activator protease (rLOPAP) of claim 72 as a coagulation time (CT) prolonging agent lasting for long periods.

125. USE of the recombinant prothrombin activator protease (rLOPAP) obtained according to the process of claim 76 as a coagulation time (CT) prolonging agent lasting for long periods.
